(19) United States
(12) Patent Application Publication
(10) Pub. No.: US 2015/0307620 A1
(43) Pub. Date: Oct. 29, 2015

(54) LINKED IMMUNOTHERAPEUTIC AGONISTS THAT COSTIMULATE MULTIPLE PATHWAYS

(71) Applicant: UNIVERSITY OF CONNECTICUT, FARMINGTON, CT (US)

(72) Inventors: ANTHONY T. VELLA, SIMSBURY, CT (US); ADAM J. ADLER, WEST HARTFORD, CT (US)

(21) Appl. No.: 14/687,422

(22) Filed: Apr. 15, 2015

Related U.S. Application Data
(60) Provisional application No. 61/980,231, filed on Apr. 16, 2014.

(51) Int. Cl. C07K 16/28

(52) U.S. Cl. CPC C07K 16/2896; C07K 16/2812; C07K 16/2815; C07K 2317/30; A61K 2039/505

(57) ABSTRACT

Described herein are modified immunotherapeutic agents including a first monoclonal antibody covalently linked to a second monoclonal antibody generating a single new immunotherapeutic agent. The first and second monoclonal antibodies stimulate different anti-tumor pathways. Advantageously, the modified single immunotherapeutic agent is capable of activating both anti-tumor pathways. Also included herein are methods of treating cancer with the modified immunotherapeutic agents.

### CROSS-REFERENCE TO RELATED APPLICATIONS

This application claims priority to U.S. Provisional Application 61/980,231 filed on Apr. 16, 2014, which is incorporated herein by reference in its entirety.

### FIELD OF THE DISCLOSURE

The present disclosure is related to modified immunotherapeutic agents and methods of treating diseases such as cancer with the modified immunotherapeutic agents.

### BACKGROUND

While great progress has been made in diagnosing cancer, there has been a lag in the development of new treatments to target tumors. Cancer immunotherapy provides new hope for treatment of patients with advanced disease. In general, the concept of immunotherapy is based on the principal of stimulating T cells through various pathways or by removing inhibitory signals to elicit powerful T cell responses directed specifically against tumors. What is needed are improved agents for cancer immunotherapy.

Melanoma, for example, has a devastating impact on public health in this and many other countries. This generates a massive burden on the health care system since many cancers are not diagnosed early and result in the most frightening aspect of cancer, which is metastatic disease. Metastatic melanoma can grow in the lung and other sites. Therefore, treatment for metastatic disease is vitally important for prolonging and enhancing the quality of life.

In addition, cost effectiveness is a major consideration in cancer immune therapies. An excellent example of this issue is Provenge®, a vaccine developed to treat advanced prostate cancer patients that was the first immune-based cancer therapy to receive FDA approval. Provenge® is a personalized therapy that involves isolation and in vitro propagation of patient-derived dendritic cells that are pulsed with tumor antigen and then re-infused back into the patient. Although Provenge® has been an important proof-of-principal and milestone in the development of cancer immunotherapies, its cost-ineffectiveness will likely preclude it from becoming a standard-of-care treatment. It is preferred to develop agents that do not utilize any patient-derived material, and rather that are analogous to off-the-shelf therapeutics such as TNF blockers that have become a standard-of-care treatment for severe inflammatory bowel disease and Ipilimumab (anti-CTLA-4) that has recently been FDA approved for melanoma.

In addition, it is very unlikely that one treatment or therapy will work for all types of cancers or even with a single type of cancer. This point is very clearly made in the recent advances and uses of biologics for treatment of human inflammatory based diseases of the joints and bowel where TNF blockers are indeed a major success story for modern biomedical medicine, but not all patients will respond positively to this treatment. Thus, there is much more to be gained by continually finding new and innovative ways to modulate the immune system. This is particularly true for cancer and even the lay press has popularized the idea that cancer is not a single disease but many different diseases. Thus, there is a need for approaches to add to the armamentarium for fighting cancer through a personalized approach, which is an emerging concept in cancer treatment.

### BRIEF SUMMARY

In one aspect, a modified immunotherapeutic agent comprises a first monoclonal antibody covalently linked to a second monoclonal antibody, wherein the first and second monoclonal antibodies stimulate different anti-tumor pathways, and wherein the modified immunotherapeutic agent becomes a single new agent capable of activating both anti-tumor pathways.

In another aspect, included herein is a method of treating cancer by administering the modified immunotherapeutic agent described herein.

In yet another aspect, a pharmaceutical composition comprises a modified immunotherapeutic agent and a pharmaceutically acceptable excipient.

### BRIEF DESCRIPTION OF THE DRAWINGS

FIG. 1 is a schematic of a modified immunotherapeutic agent according to the present disclosure.

FIG. 2 is a schematic of mixed signaling by a modified immunotherapeutic agent according to the present disclosure.

FIG. 3 shows the production of a crosslinked hetero-conjugate of anti-OX40 and anti-4-1BB (hetero-dimer indicated by arrow).

FIG. 4 shows the production of a hetero-conjugate of anti-OX40 and anti-4-1BB produced using click chemistry (hetero-dimers and hetero-tetramers indicated by green arrows). The final agent is a single drug capable of stimulating 2 different pathways.

FIGS. 5A, 5B and 5C show an analysis of a hetero-conjugate of anti-OX40 and anti-4-1BB produced using click chemistry. FIG. 5A shows the FPLC gel filtration fractionation profile, FIG. 5B shows the gel filtration calibration curve generated using size standards, and FIG. 5C shows an analysis of individual fractions by SDS-PAGE.

FIG. 6 shows granzyme B expression in response to the purified and non-purified hetero-conjugates and non-conjugated dual costimulation in CD8 T cells.

FIG. 7 shows granzyme B expression in response to the purified and non-purified hetero-conjugates and non-conjugated dual costimulation in CD4 T cells.

FIG. 8 shows an FPLC gel filtration run of anti-OX40 and anti-4-1BB hetero-conjugate (referred to hereafter as “OrthomAb”) (top panel) and an SDS PAGE analysis of the OrthomAb fractions (bottom left panel). Fractions #7-12 (refer to red box) were pooled, concentrated and subjected to a second FPLC run. The bottom right panel shows the final highly enriched OrthomAb product. Please note that this production run is distinct from that shown in FIG. 4, both of which are representative of several runs.

FIG. 9 shows the in vivo costimulatory potential of FPLC-enriched OrthomAb as evidenced by flow cytometry analysis of the potential of antigen-primed CD4 T cells to express the effector cytokines IFN-γ and TNF-α. The OrthomAb preparations A through E represent pools of differently sized hetero-conjugates with pool A comprised mostly of hetero-dimers and B through E containing progressively larger sized hetero-conjugates. “IsomAb” is the control hetero-conjugate produced using monoclonal antibodies with the same isotype (Fc region) as the anti-OX40 and anti-4-1BB antibodies but irrelevant variable domains (i.e., binding specificities).

FIG. 10 shows in vivo therapeutic activity of FPLC-enriched OrthomAb in the highly aggressive B16-F10 mouse melanoma model. Mice were inoculated intradermally with B16-F10 tumor cells, and three days later when tumors had become established (visually detectable) treated with OrthomAb or control IsomAb. Mice received a second (booster) treatment three days following the first treatment. OrthomAb slowed tumor growth up until Day 12 (six days following the final treatment).

FIG. 11 is a schematic of the synthesis of F(ab′)2 Orthus constructs.

The above-described and other features will be appreciated and understood by those skilled in the art from the following detailed description, drawings, and appended claims.

### DETAILED DESCRIPTION

The novel therapeutic approach of modified immunotherapeutic agents described herein is termed Orthus. Orthus capitalizes on the inventors' experience studying T cell costimulation and approaches that accentuate tumor cell killing. It has been demonstrated that triggering two costimulatory pathways is more powerful than triggering one pathway. The novel Orthus approach employs a single reagent that can trigger two separate pathways, such as two T cell costimulatory pathways, rather than using two separate reagents to separately stimulate these pathways. Results generated over several years have shown that stimulating the two costimulatory pathways through co-administration of the two separate reagents is synergistic over triggering single pathways. The essential premise of Orthus is to simultaneously stimulate two pathways such as two costimulatory pathways in direct physical proximity to each other rather than triggering them separately. Orthus may provide a paradigm shift in immunotherapy treatment of cancer.

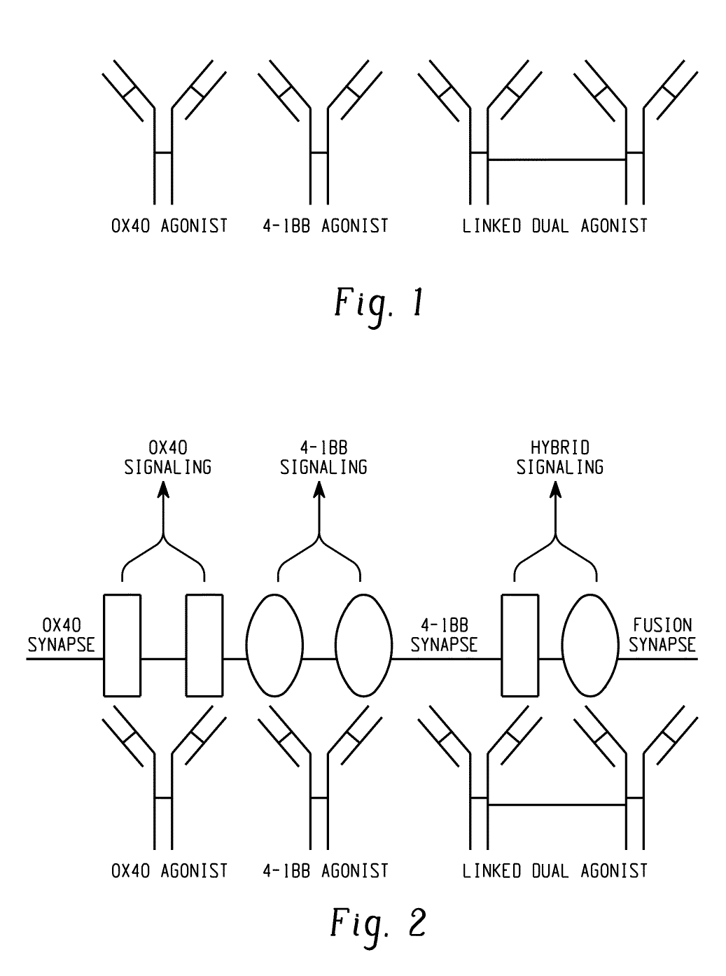

A modified immunotherapeutic agent comprises a first monoclonal antibody covalently linked to a second monoclonal antibody, illustrated in FIG. 1, wherein the first and second monoclonal antibodies each stimulate different anti-tumor pathways. The first and second monoclonal antibodies are covalently linked such that the binding activities of both the first and second antibodies are maintained, that is, the modified immunotherapeutic agent is capable of activating both anti-tumor pathways.

In an aspect, the first monoclonal antibody, the second monoclonal antibody, or both, is an agonist of a T cell costimulatory pathway. When the first and second monoclonal antibodies are both agonists of T cell costimulatory pathways, the first and second monoclonal antibody are agonists of different T cell costimulatory pathways. In one aspect, the first or second monoclonal antibody is an agonist of a costimulatory pathway receptor selected from CD134 (OX40), CD137 (4-1BB), CD28, GITR, CD27, CD70, ICOS, RANKL, TNFRSF25 (DR3), CD258 (LIGHT), CD40, HVEM, and the like.

In an aspect, the first monoclonal antibody is an agonist of a costimulatory pathway and the second monoclonal antibody is an agonist of a checkpoint inhibitor. Exemplary checkpoint inhibitor receptors include CTLA-4, PD-1, TIM-3, LAG-3 and CD55.

Anti-OX40 antibodies are described, for example, in U.S. Pat. Nos. 8,614,295; 7,501,496; and 8,283,450, incorporated herein by reference in their entirety for the disclosure of anti-OX40 antibodies.

Anti-4-1BB antibodies are described, for example, in U.S. Pat. Nos. 6,569,997; 6,974,863; and 8,137,667, incorporated herein by reference in their entirety for the disclosure of anti-4-1BB antibodies.

Anti-CD28 antibodies are described, for example, in U.S. Pat. Nos. 7,585,960; 8,334,102, and 7,723,482, incorporated herein by reference in their entirety for the disclosure of anti-CD28 antibodies.

Anti-GITR antibodies are described, for example, in U.S. Pat. Nos. 7,812,135 and 8,388,967, incorporated herein by reference in their entirety for the disclosure of anti-GITR antibodies.

Anti-CD27 antibodies are described, for example, in U.S. Pat. No. 8,481,029, incorporated herein by reference in its entirety for the disclosure of anti-CD28 antibodies.

Anti-CD70 antibodies are described, for example, in U.S. Pat. Nos. 8,337,838; 8,124,738; and 7,491,390, incorporated herein by reference in their entirety for the disclosure of anti-CD70 antibodies.

Anti-ICOS antibodies are described, for example, in U.S. Pat. Nos. 7,521,532 and 8,318,905, incorporated herein by reference in their entirety for the disclosure of anti-ICOS antibodies.

Anti-RANKL antibodies are described, for example, in U.S. Pat. Nos. 7,411,050; 8,414,890, and 8,377,690, incorporated herein by reference in their entirety for the disclosure of anti-RANKL antibodies. An exemplary anti-RANKL antibody is denosumab.

Anti-TNFRSF25 (DR3) antibodies are described, for example, in U.S. Patent Publication Nos. US20130330360, and US20120014950 incorporated herein by reference in their entirety for the disclosure of anti-DR3 antibodies.

Anti-CD258 (LIGHT) antibodies are described, for example, in U.S. Patent Publication Nos. US20130315913 and US20090214519, incorporated herein by reference in their entirety for the disclosure of anti-LIGHT antibodies.

Anti-CD40 antibodies are described, for example, in U.S. Pat. Nos. 8,669,352; 8,637,032; 8,591,900; 8,492,531; 8,388,971; 8,303,955; 7,790,166; 7,666,422; 7,563,442; 7,537,763; and 7,445,780, incorporated herein by reference in their entirety for the disclosure of anti-CD40 antibodies.

Anti-HVEM antibodies are described, for example, in U.S. Pat. Nos. 6,573,058, and 8,440,185, incorporated herein by reference in their entirety for the disclosure of anti-HVEM antibodies.

Anti-CTLA-4 antibodies are described, for example, in U.S. Pat. Nos. 8,142,778; 8,017,114; 7,132,281; 7,109,003; 6,984,720; and 6,682,736, incorporated herein by reference in their entirety for the disclosure of anti-CTLA-4 antibodies.

Anti-PD-1 antibodies are described, for example, in U.S. Pat. Nos. 8,354,509; 8,088,905; 8,008,449; and 7,488,802, incorporated herein by reference in their entirety for the disclosure of anti-PD-1 antibodies.

Anti-TIM-3 antibodies are described, for example, in U.S. Pat. Nos. 8,552,156 and 8,101,176, incorporated herein by reference in their entirety for the disclosure of anti-TIM-3 antibodies.

Anti-LAG-3 antibodies are described, for example, in U.S. Pat. No. 6,143,273, US20110150892, and US20110070238, incorporated herein by reference in their entirety for the disclosure of anti-LAG-3 antibodies.

Anti-CD55 antibodies are described, for example, in U.S. Pat. No. 8,034,902, incorporated herein by reference in its entirety for the disclosure of anti-CD55 antibodies.

In one aspect, a modified immunotherapeutic agent comprises a first monoclonal antibody covalently linked to a second monoclonal antibody, wherein the first monoclonal antibody primarily activates CD4 T cells and the second monoclonal antibody primarily activates CD8 T cells. By “primarily activates CD4 T cells”, it means that a monoclonal antibody activates CD4 T cells to a greater extent than CD8 T cells. By “primarily activates CD8 T cells”, it means that a monoclonal antibody activates CD8 T cells to a greater extent than CD4 T cells.

In one aspect, a modified immunotherapeutic agent comprises two monoclonal antibodies, anti-OX40 and anti-4-1BB, covalently linked into a single agent. The first pathway, OX40 (CD134), leads to robust CD4 T cell activation. These CD4 T cells develop the capacity to secrete cytokines, migrate into peripheral tissues and maintain survival characteristics. The second pathway, 4-1BB (CD137), primarily activates CD8 T cells that acquire similar functional characteristics as the CD4 T cells, which include cytokine synthesis, migratory ability and survival. Further, 4-1BB is known to activate the innate immune system, which may provide a distinct advantage over agents that only activate T cells. For example, 4-1BB agonists activate dendritic cells and NK cells, both of which are known to play an important role in tumor immunity. A wealth of data has been generated over the last two decades demonstrating the power of these costimulatory agonists in eradicating tumors in rodents. Previous work has demonstrated that dual administration of OX40 plus 4-1BB agonists is superior compared to singly applied agonists in controlling tumor growth. Chemically linking the agonist monoclonal antibodies together so that they work in proximity and in concert with each other contrasts with prior usage of dual agonists where their sites of action may not necessarily be in physical or temporal proximity.

One of the intriguing aspects of dual costimulation is the generation of CD4 T cells that take on characteristics of CD8 T cells. Impressively, dual-costimulated CD4 T cells acquire the ability to be cytotoxic through their expression of intracellular granzymes and ability to directly kill tumor cells. In a recent report, the ability of dual-costimulated CD4 T cells to kill tumor cells in vitro and at least help control tumor growth in vivo was demonstrated. To be more precise, dual costimulation therapy, stimulation with 2 separate agents, was effective in programming CD4 T cells to limit B16 melanoma growth in the absence of CD8 T cells. In fact, there was a trend by dual costimulation therapy to limit tumor growth in the absence of any T cells, further demonstrating the power of this approach.

Without being held to theory, it is hypothesized that by chemically linking OX40 and 4-1BB agonists together, for example, the action of the dual-agonist will be focused to cells that either co-express both receptors (i.e., activated T cells) or that make direct contact with each other (e.g., 4-1BB-expressing dendritic cells that present antigen to OX40-expressing T cells). Also, T cell responses require lower doses of dual costimulation than an equivalent response mediated by either reagent alone. It is thus predicted that at a low dose the dual-agonist will preferentially act on the relevant cells critical for anti-tumor immunity while avoiding activation of cell types that can only facilitate adverse effects.

The first and second monoclonal antibodies are covalently linked using methods known in the art such as click chemistry. “Click chemistry” is defined as a chemical reaction involving molecular building blocks that selectively and covalently bond or “click” together. A “cycloaddition” reaction is defined as a type of click chemistry reaction. One embodiment of click chemistry utilizes a 1+3-dipolar cycloaddition reaction of azide and alkyne functional groups, otherwise referred to as a [3+2] cycloaddition reaction. Other embodiments may involve other reactions including, for example, the Diels-Alder [4+2] cylcoaddition reaction between a diene and a dienophile.

The click chemistry approach was originally conceived as a method to rapidly generate complex substances by joining small subunits together in a modular fashion. Various forms of click chemistry reaction are known in the art, such as the Huisgen 1,3-dipolar cycloaddition copper catalyzed reaction which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

In one aspect, covalent linking is crosslinking, such as crosslinking with sulfo-SMCC (sulfosuccinimidyl-4-[N-maleimidomethyl]cyclcohexane-1-carboxylate) or SATA (N-Succinimidyi-S-Acetyl-Thioacetate).

In one aspect, the first and second antibodies may be covalently linked through the Fc region.

The potential therapeutic advantages of using the Orthus approach are the following: 1) Preliminary data using non-linked agonists demonstrates synergism in programming T cell effector and tumoricidal function compared to using single agonists. Without being held to theory, it is believed that chemically linking the two agonists together will result in a stronger blast of costimulation. The rationale is that ensuring that both costimulatory receptors are engaged next to each other on the same cell will result in clustering of the receptors and associated intracellular signaling pathways and hence a more robust (and perhaps artificial) anti-tumor T cell response. For example, clustering OX40 within a single synapse triggers a somewhat different intracellular signaling pathway than engaging 4-1BB, and thus combining these pathways within a single synapse will result in a stronger response that will allow for the use of lower dosages of agonists to achieve beneficial therapeutic effects while limiting toxicities that might result from using higher dosages. This process is referred to as “synapse fusion”. Thus, normally one receptor is triggered by one ligand to initiate a particular intracellular signaling pathway, but in this case triggering both pathways in mixed proximity using a single bivalent ligand will lead to a hybrid signal (FIG. 2).

In one aspect, the modified immunotherapeutic agents described herein are used in cancer therapy, particularly in humans. The modified immunotherapeutic agents can be used to treat advanced and metastatic cancers, as well as in the prevention of cancer.

As used herein, cancer includes cancer of the skin (melanoma), solid organ based cancers (e.g., those arising from the kidney, pancreas, lung, intestinal, prostate, breast, liver, etc.) as well as hematological cancers such as lymphomas for which there is a pressing need for novel therapies. In a specific embodiment, the melanoma cancer progresses because of alterations in the T cell response to the melanoma. Specifically, during the progression of melanoma it is well documented that melanoma-specific T cells expand in number, and can be detected using melanoma antigen tetramer analysis. Additionally, immune modulators that block the T cell checkpoint inhibitors PD-1 and CTLA-4 have shown promise in the clinic, thus demonstrating that melanoma is amenable to immunotherapy.

Specifically with regard to melanoma, although melanoma patients harbor expanded tumor-reactive T cells, they are non-functional. Without being held to theory, it is hypothesized that an advantage of modified immunotherapeutic agent as described herein is the potential to re-awaken these anergic T cells in the tumor micro-environment. This might contrast with the checkpoint inhibitors mentioned above, but this is currently untested. Nevertheless, it is very well documented that these costimulatory agonists can resuscitate anergic T cells in tumor models and functional immune studies. These data suggest that resuscitating anergic melanoma-specific T cells will address an un-met need in the therapy of melanoma patients that have progressed to metastatic disease. An important consideration in the development of T cell-based cancer therapies is the necessity of the specific T cells to traffic into tumors located throughout the body. Thus, another advantage of the Orthus approach is that (at least in model systems) dual-costimulated T cells traffic into virtually all organs. A specific example is that dual-costimulated T cells can enter and expand in the lung that is a common site for metastatic melanoma. In sum, this approach resuscitates anergic tumor-specific T cells and allows them to scan all parts of the body where metastatic cells are likely to hide.

The phrase “effective amount,” as used herein, means an amount of an agent which is sufficient enough to significantly and positively modify symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

The amount of modified immunotherapeutic agent effective for any indicated condition will, of course, vary with the individual subject being treated and is ultimately at the discretion of the medical or veterinary practitioner. The factors to be considered include the condition being treated, the route of administration, the nature of the formulation, the subject's body weight, surface area, age and general condition, and the particular compound to be administered. In general, a suitable effective dose is in the range of about 0.1 to about 4 mg/kg body weight per day, preferably in the range of about 1 to about 2.5 mg/kg per day. The total daily dose may be given as a single dose three times a week, which is considered as one treatment cycle. Dosages or other treatment cycles above or below the range cited above may be administered to the individual patient if desired and necessary.

In one aspect, the dose of the first and second monoclonal antibodies in the modified immunotherapeutic agent is reduced compared to the dose for each antibody administered individually. Without being held to theory, it is believed that the modified immunotherapeutic agent described herein will provide a synergistic effect of stimulating two pathways, and that the effect will be significantly greater than that observed when the two agents are administered as separate reagents.

As used herein, “pharmaceutical composition” means therapeutically effective amounts of the modified immunotherapeutic agent together with a pharmaceutically acceptable excipient, such as diluents, preservatives, solubilizers, emulsifiers, and adjuvants. As used herein “pharmaceutically acceptable excipients” are well known to those skilled in the art.

In all likelihood dosages will be delivered intravenously similar to current biologics. Nevertheless, it is possible that tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

The active ingredient may be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The invention is further illustrated by the following non-limiting examples.

### EXAMPLES

### Example 1

### Chemical Linking of Antibodies Using Crosslinking

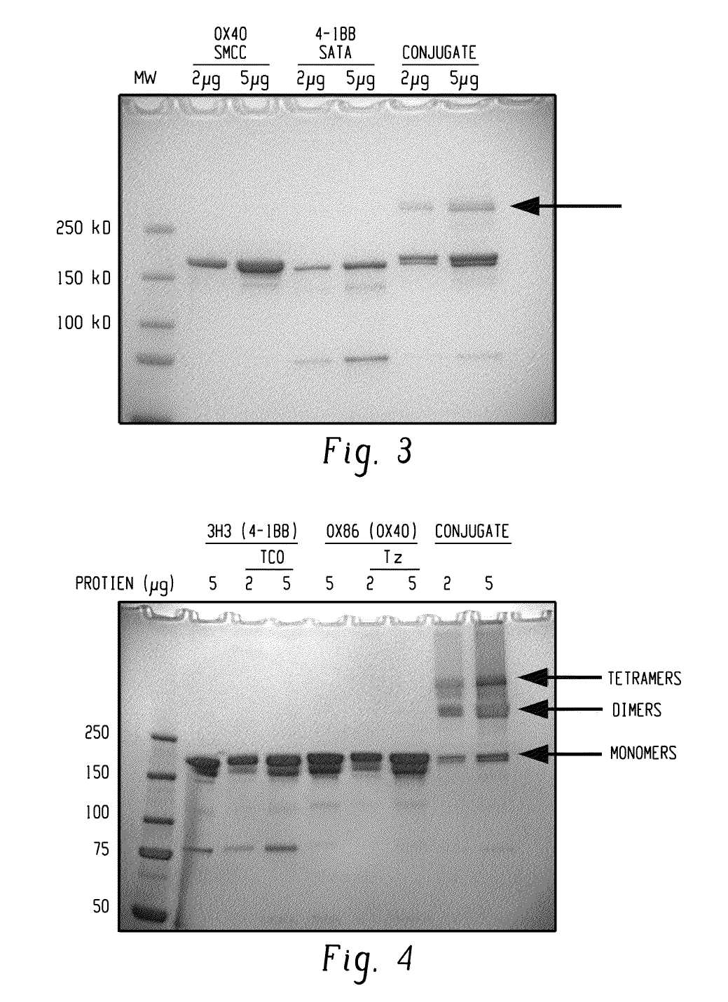

Mouse monoclonal anti-OX40 to anti-4-1BB antibodies were chemically linked using the “male-female” cross-linking agents sulfo-SMCC and SATA. FIG. 3 shows that when anti-OX40 modified with SMCC is mixed with anti-4-1BB modified with SATA a species forms that has a molecular weight consistent with the additive weight of the individual monoclonal antibody species (approximately 400 kD, indicated by the arrow). This band representing the antibody hetero-conjugate was observed in 3 separate studies that followed similar chemical modification and conjugation conditions.

### Example 2

### Chemical Linking of Antibodies Using Click Chemistry

The click chemistry couplers Trans-Cyclooctene-PEG4-NHS ester (TCO) and Tetrazine-PEG5-NHS ester (Tz) (coupling kit available from Click Chemistry Tools, Scottsdale, Ariz.) are separately attached to the 4-1BB and OX40 mAbs, respectively, and then the two coupled mAbs are incubated together to form hetero-conjugates. As shown in FIG. 4, when the anti-mouse mAbs to 4-1BB (clone 3H3, available from BioXcell, West Lebanon, N.H.) coupled to TCO and OX40 (clone OX86, available from BioXcell) coupled to Tz are incubated together hetero-conjugates form whose molecular weights estimated from SDS-PAGE gel electrophoresis are consistent with dimeric and tetrameric species (refer to arrows).

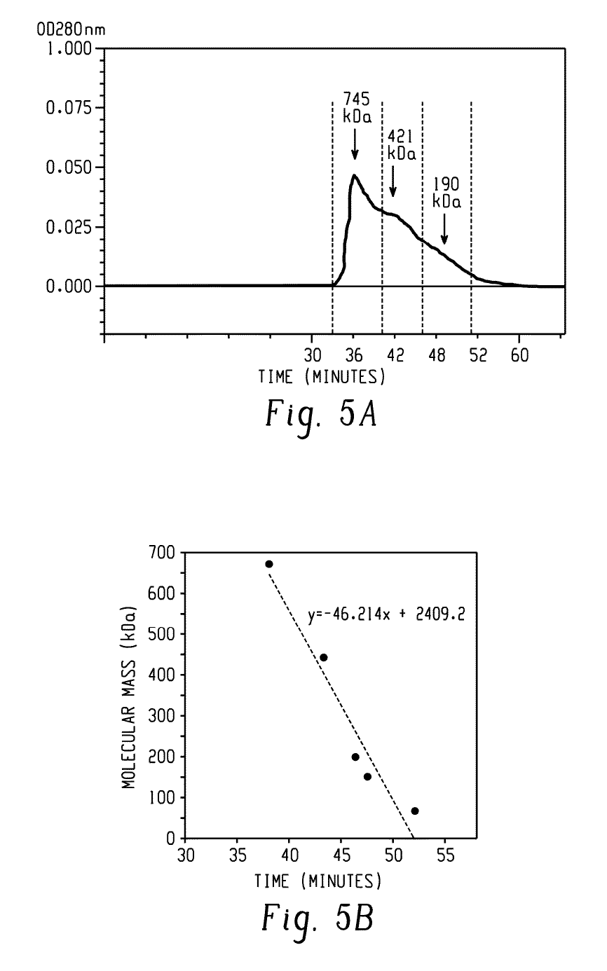

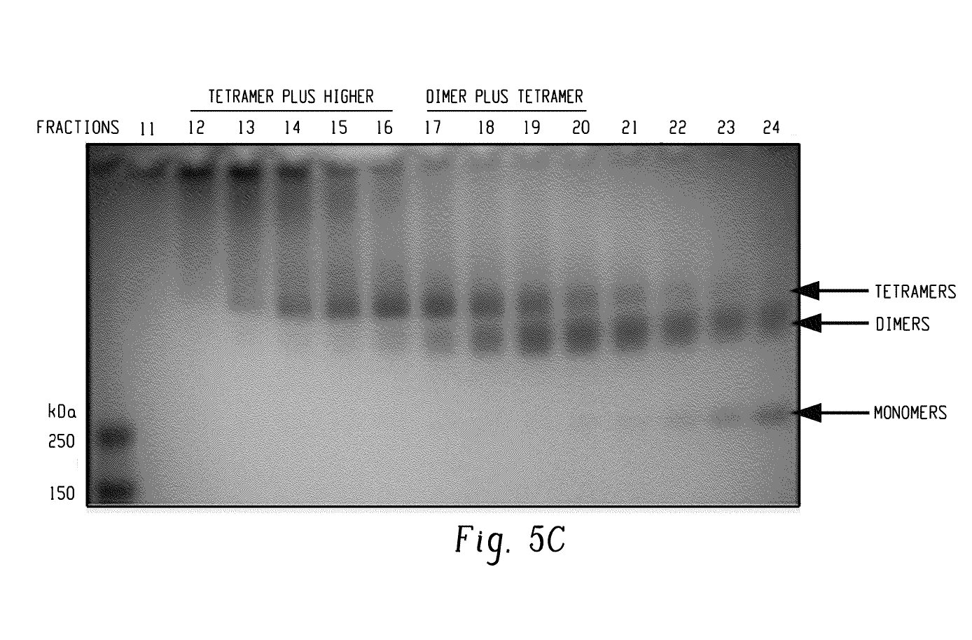

The hetero-conjugates were then separated via gel filtration chromatography using Sephacryl 300 (Sigma, St. Louis, Mo.) (FIG. 5A shows the fractionation profile of the OX40-4-1BB hetero-conjugates, FIG. 5B shows the calibration curve generated from size standards). Individual fractions were then analyzed by SDS-PAGE (BioRad, Hercules, Calif.) (FIG. 5C), and fractions 12-16 corresponding tetramer and higher molecular weights species (referred to hereafter as “purified tetramer”) were pooled and fractions 17-20 corresponding to dimers plus tetramers were pooled (referred to hereafter as “purified dimer+tetramer”).

### Example 3

### In Vitro Costimulation Assay

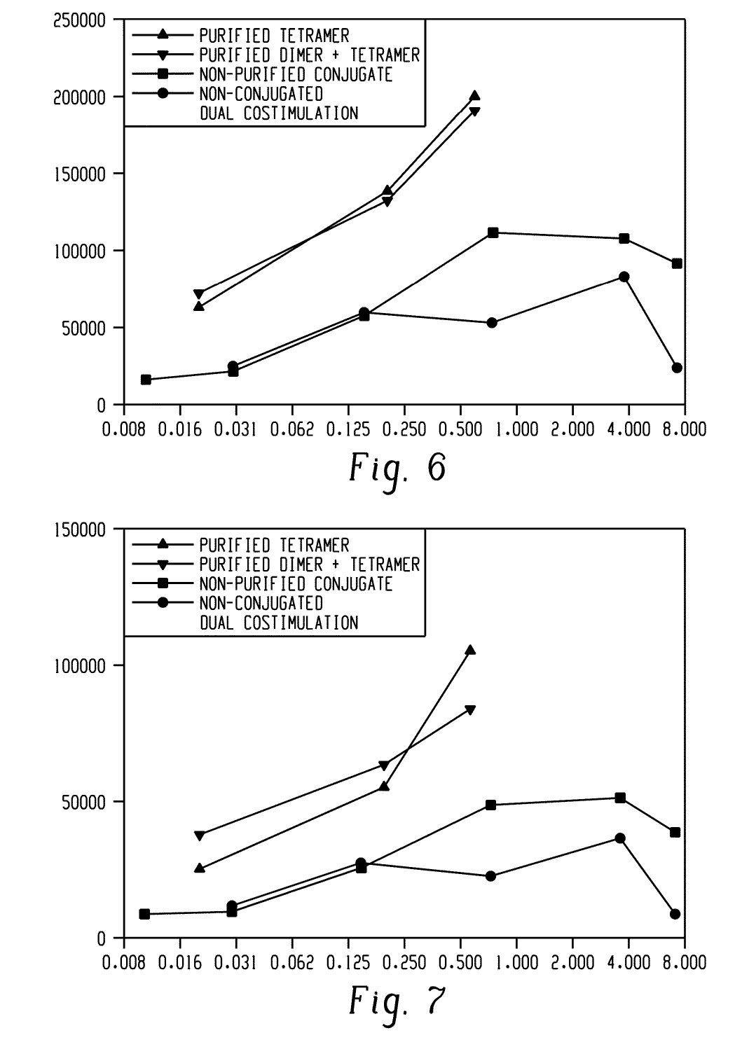

The purified conjugate fractions from Example 2 were then tested in an in vitro costimulation assay (FIGS. 6 and 7). Specifically, mouse CD4⁺ and CD8⁺ T cells contained within pooled spleen plus lymph node preparations were stimulated with anti-CD3 mAb (eBiosciences, San Diego, Calif.) at a dosage (0.01 μg/ml) that only elicits partial activation along with titrated dosages of purified hetero-conjugate fractions. As controls, non-fractionated hetero-conjugates and non-conjugated OX40 plus 4-1BB agonists (referred to hereafter as “dual costimulation”) were also tested. Following 48 hours, the CD4 and CD8 T cells were analyzed by flow cytometry (UCHC Flow cytometry facility) to measure expression of the cytotoxic molecule granzyme B (eBiosciences), which is perhaps the most accurate marker of T cell killing potential. As shown in FIG. 6, both the purified tetramer and dimer+tetramer fractions elicited substantial expression of granzyme (expressed in the graph as mean fluorescence intensity or MFI) in CD8 T cells at a low dosage (0.016 μg/ml). Importantly, approximately 10-fold greater amounts of non-purified conjugate and non-conjugated dual costimulation were required to elicit comparable granzyme B expression. Further, even at the highest concentrations tested, non-purified conjugate and non-conjugated dual costimulation elicited granzyme B expression levels that were substantially lower than those achieved with lower concentrations of purified hetero-conjugates.

As shown in FIG. 7, CD4 T cells showed as a similar pattern of granzyme B expression in response to the purified and non-purified hetero-conjugates and non-conjugated dual costimulation. Thus, purified hetero-conjugates elicited greater amounts of granzyme B expression, and at lower concentrations. Similar results were observed in two independent experiments.

### Example 4

### Optimization of conjugate manufacture

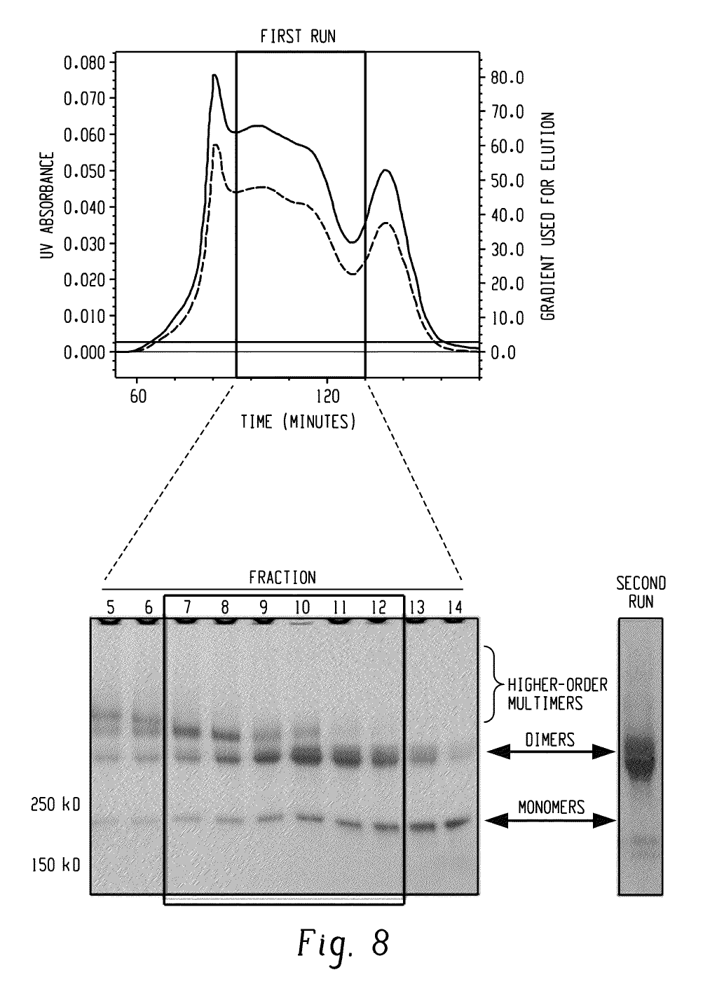

Using the Click Chemistry coupling technique described in Example 2 and FIG. 4, anti-OX40/anti-4-1BB hetero-conjugates (referred to hereafter as “OrthomAb”) as well as control isotype control hetero-conjugates (referred to hereafter as “IsomAb”) were generated. Both hetero-conjugate preparations were then subjected to FPLC gel filtration using Sephacryl 300 beads to separate the different sized conjugates from each other as well as from the residual monomers (as described in FIG. 5). A typical FPLC run is shown below in the top panel of FIG. 8 where protein UV absorbance shown on the leftmost axis is plotted relative to run time. Individual fractions were then analyzed by SDS PAGE (bottom left panel). OrthomAb fractions containing mostly dimers (as well as residual monomers and larger hetero-conjugates, fractions 7-12) were pooled from several preparations, concentrated and then subjected to a second FPLC run. As shown in the bottom right panel of FIG. 8, this produced a final product that was highly enriched for OrthomAb.

### Example 5

### In Vivo Costimulatory Potential

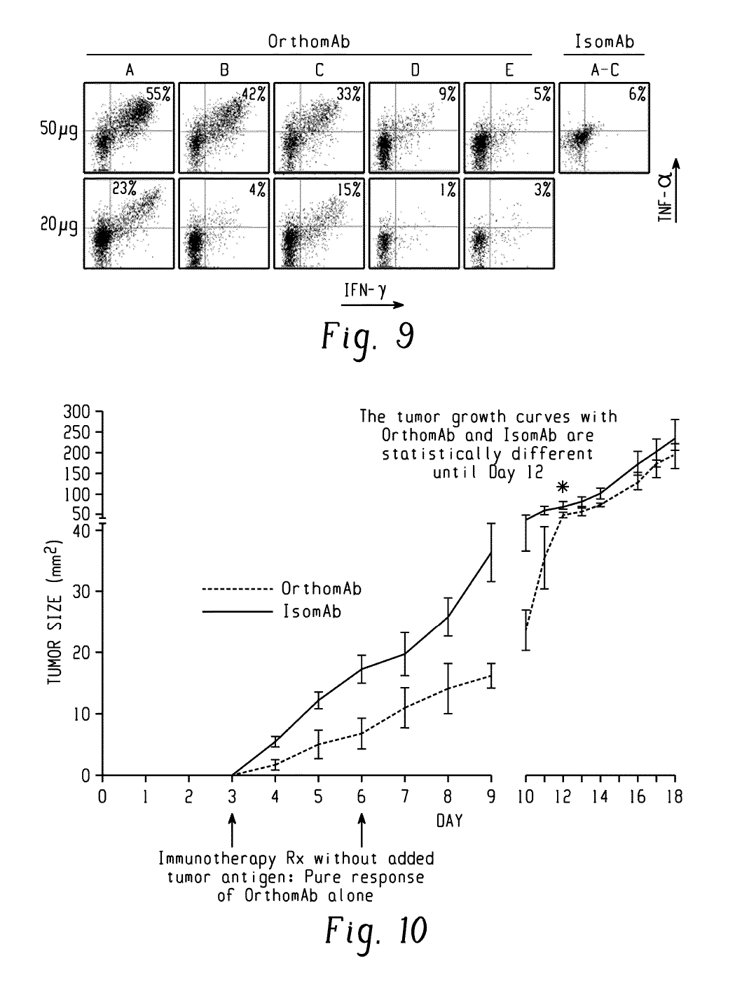

A separate OrthomAb preparation subjected to a single FPLC run was pooled and concentrated into 5 tubes labeled “A” through “E”. Pool A contained predominantly dimers, while Pools B through E contained progressively larger sized conjugates (data not shown). Each pool was then tested in an adoptive T cell transfer model in which T cell receptor transgenic CD4 T cells are activated in vivo with cognate soluble antigenic peptide, but only gain functional capacity when concomitantly provided costimulation. As shown by flow cytometry (FIG. 9), Pool A (containing mostly dimers) programmed antigen-responding CD4 T cells to express the effector cytokines IFN-γ and TNF-α in a dose-dependent manner (i.e., more cytokine with 50 μg than with 20 μg).

This costimulation capacity gradually diminished with OrthomAb pools containing progressively larger conjugates to the point that the activity of Pool E (containing the largest conjugates) approached the background observed with 50 μg of control IsomAb composed of the equivalent of Pools A-C. Similarly, Pool A also programmed the T cells to express the greatest amounts of the transcription factor Eomesodermin as well as the high affinity IL-2 receptor CD25 (not shown) which both play critical roles in programming various T cell effector functions. Taken together, these data suggest that the smaller OrthomAb conjugates (i.e., dimers) may be the most potent.

### Example 6

### In Vivo Cancer Therapeutic Potential of Orthus

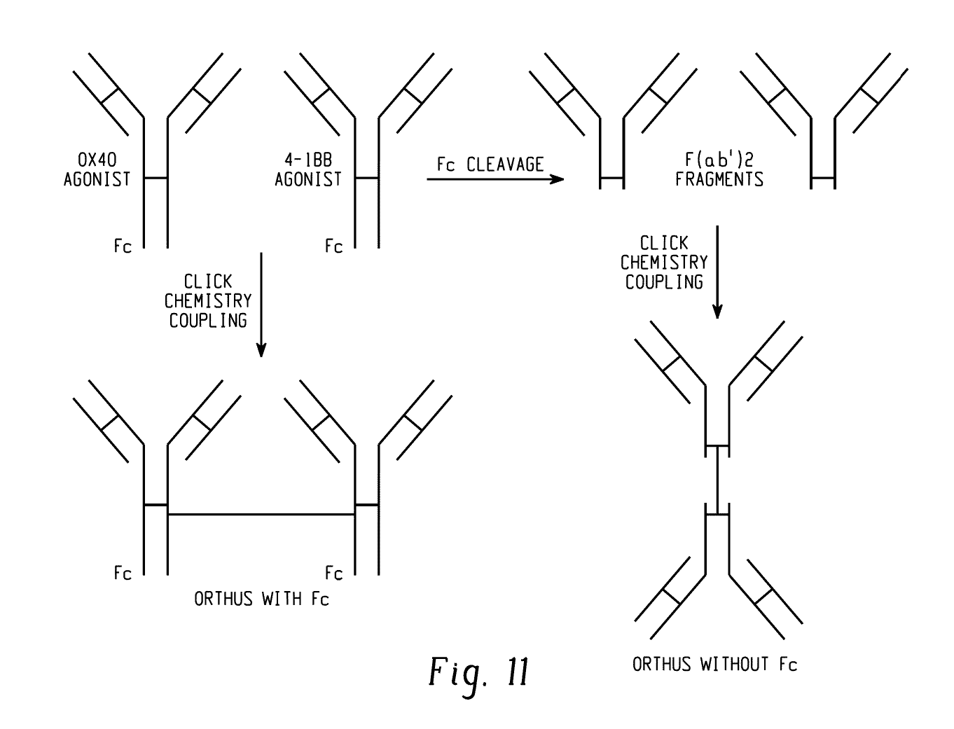

The highly aggressive B16-F10 melanoma model was used to test the therapeutic potential of OrthomAb. Once established, this tumor is notoriously difficult to treat, and thus represents a rigorous pre-clinical model. In the experiment shown in FIG. 10, C57BL/6J mice where transplanted intradermally with 1×10⁵ B16-F10 tumor cells. Three days later when the tumors had become established (i.e., visually detectable mass), the mice were treated intraperitoneally with 150 μg OrthomAb or control IsomAb (both prepared using the method described in FIG. 8). Mice received a second (booster) treatment on Day 6 (i.e., three days following the first treatment). Tumors were measured at the indicated times using calipers and multiplying perpendicular diameters to calculate surface areas in millimeters squared.

Despite the small sample sizes (4 mice received OrthomAb and 5 received control IsomAb), there was a statistically significant reduction (p<0.05) in the tumor growth curve (measured using area under the curve analysis) of OrthomAb compared to IsomAb-treated mice beginning the day following the initial treatment (Day 4) until Day 12 (6 days following the booster treatment). Beyond this time, tumor growth in the OrthomAb-treated mice caught up to the controls. Thus, OrthomAb's therapeutic activity tracks with its timing and duration of administration.

### Example 7

### Reducing Toxicities

Costimulatory agonists, that have until now been administered only as monomers in therapeutic settings, require an intact Fc domain as well as the presence of accessory cells expressing Fc receptors (FcR) for therapeutic efficacy against tumors. This is explained by the necessity to co-localize multiple costimulatory receptors to elicit down-stream signaling. In particular, costimulatory receptors belonging to the TNFR superfamily (including OX40 and 4-1BB) must be trimerized to initiate down-stream signaling. Given that an individual agonist antibody can only bind two receptor subunit monomers, FcR-mediated antibody clustering should co-localize three or more receptor subunit monomers and thus trigger down-stream signaling. On the other hand, engagement of FcR on certain innate cell types may elicit inflammatory responses that mediate non-therapeutic toxic side effects. For instance, engagement of these FcR-expressing innate cells may trigger the innate cells themselves to release large amounts of inflammatory mediators that when present at high systemic levels cause adverse events. An approach to minimize these potential toxicities while simultaneously targeting costimulation to the therapeutically-relevant tumor-specific T cells will be to generate conjugates using costimulatory agonist F(ab′)2 fragments that lack Fc domains. Thus, while a F(ab′)2 fragment without Fc can only cross-link 2 receptor subunit monomers in the absence of FcR-mediated cross-linking and thus fail to deliver effective costimulation, a hetero-dimer of F(ab′)2 fragments lacking Fc has the potential to simultaneously co-localize 2 OX40 along with 2 4-1BB receptor subunit monomers on the same T cell, and higher order conjugates could co-localize an even greater number of receptor subunit monomers. Conjugation of agonists lacking Fc may therefore enable sufficient receptor subunit monomer co-localization to elicit therapeutic effects while avoiding engagement of therapeutically-irrelevant but toxicity-producing FcR-expressing innate cells. Another potential advantage of F(ab′)2 Orthus would be its reduced overall size (compared to Fc-intact Orthus) which may enhance its ability to penetrate solid tumors and engage tumor-infiltrating effector T cells directly within the tumor microenvironment, and prevent its trapping by FcRs expressed on immune cells. Orthus without Fc will be manufactured by first removing the Fc domains from the OX40 and 4-1BB agonists via pepsin cleavage, and then conjugating the resulting F(ab′)2 fragments using the click chemistry methodology described above for the Fc-containing agonists (FIG. 11). The F(ab′)2 monomers and hetero-conjugates will be tested for their in vivo ability to both costimulate T cells (using the assay described in FIG. 9) and mediate therapeutic benefit in controlling tumor growth (using the assay described in FIG. 10). It is predicted that OX40 plus 4-1BB F(ab′)2 monomers, due to their inability to co-localize three or more receptor subunit monomers, will fail to elicit positive responses in both assays. In contrast, the F(ab′)2 hetero-conjugates are predicted to both efficiently costimulate T cells and mediate tumor growth control. Finally, compared to Fc-intact (non-modified) OX40 plus 4-1BB agonists the F(ab′)2 hetero-conjugates are also predicted to elicit production of lower systemic levels of pro-inflammatory cytokines that can be secreted by activated FcR-expressing innate cells that when present at high systemic levels can mediate toxic adverse events. This will be tested by measuring serum levels of relevant pro-inflammatory cytokines such as TNF-α, IL-1β and IL-6. Specifically, it is predicted that F(ab′)2 hetero-conjugates will elicit lower serum levels of these cytokines compared to Fc-intact agonists.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A modified immunotherapeutic agent comprising
a first monoclonal antibody covalently linked to a second monoclonal antibody,
wherein the first and second monoclonal antibodies stimulate different anti-tumor pathways, and wherein the modified immunotherapeutic agent is capable of activating both anti-tumor pathways.

2. The modified immunotherapeutic agent of claim 1, wherein the first monoclonal antibody, the second monoclonal antibody, or both, is an agonist of a T cell costimulatory pathway.

3. The modified immunotherapeutic agent of claim 2, wherein the costimulatory pathway receptor is CD134 (OX40), CD137 (4-1BB), CD28, GITR, CD27, CD70, ICOS, RANKL, TNFRSF25 (DR3), CD258 (LIGHT), CD40, or HVEM.

4. The modified immunotherapeutic agent of claim 2, wherein the first monoclonal antibody is an agonist of CD134 (OX40) and the second monoclonal antibody is an agonist of CD137 (4-1BB).

5. The modified immunotherapeutic agent of claim 1, wherein the first monoclonal antibody is an agonist of a T cell costimulatory pathway and the second monoclonal antibody is an agonist of a checkpoint inhibitor.

6. The modified immunotherapeutic agent of claim 5, wherein the costimulatory pathway receptor is CD134 (OX40), CD137 (4-1BB), CD28, GITR, CD27, CD70, ICOS, RANKL, TNFRSF25 (DR3), CD258 (LIGHT), CD40, or HVEM.

7. The modified immunotherapeutic agent of claim 5, wherein the checkpoint receptor is CTLA-4, PD-1, TIM-3, LAG-3 or CD55.

8. The modified immunotherapeutic agent of claim 1, wherein the first monoclonal antibody primarily activates CD4 T cells and the second monoclonal antibody primarily activates CD8 T cells.

9. The modified immunotherapeutic agent of claim 1, wherein the first and second monoclonal antibodies are covalently linked by click chemistry or crosslinking.

10. The modified immunotherapeutic agent of claim 9, wherein the first and second monoclonal antibodies are covalently linked through their Fc regions.

11. The modified immunotherapeutic agent of claim, wherein the first and second monoclonal antibodies are F(ab′)2 fragments without Fc domains, and wherein the F(ab′)2 fragments are covalently linked using click chemistry.

12. A method of treating cancer in a human subject, comprising administering to the human subject an effective amount of the modified immunotherapeutic agent of any one of claims 1-11.

13. The method of claim 12, wherein the cancer is advanced or metastatic cancer.

14. The method of claim 12, wherein the cancer is melanoma, kidney cancer, pancreatic cancer, prostate cancer, breast cancer, liver cancer, or lymphoma.

15. The method of claim 12, wherein the cancer is melanoma.

16. A pharmaceutical composition comprising the modified immunotherapeutic agent of claim 1 and a pharmaceutically acceptable excipient.
